(19) United States
(12) Patent Application Publication
(10) Pub. No.: US 2024/0090794 A1
(43) Pub. Date: Mar. 21, 2024

(54) ACOUSTIC SENSOR DEVICE WITH INTEGRATED GAS PROPERTY SENSORS

(71) Applicant: Covidien LP, Mansfield, MA (US)

(72) Inventors: Matthew J. Phillips, Carlsbad, CA (US); Steven J. Bredehoft, Orange, CA (US); Jeffrey P. Mansfield, Bloomington, IN (US)

(73) Assignee: Covidien LP, Mansfield, MA (US)

(21) Appl. No.: 18/459,607

(22) Filed: Sep. 1, 2023

Related U.S. Application Data
(60) Provisional application No. 63/376,009, filed on Sep. 16, 2022.

(51) Int. Cl. A61B 5/083; A61B 5/00

(52) U.S. Cl. CPC A61B 5/0836; A61B 5/4848

(57) ABSTRACT

An acoustic sensor device including a proximal end connectable to a breathing circuit to receive breathing gases; a distal end connectable to the tracheal tube; a housing, between the proximal end and the distal end, defining a lumen through which the breathing gases flow; an acoustic generator within the housing and positioned to emit acoustic pulses into the lumen; an acoustic receiver within the housing and positioned distally from the acoustic generator; and a first gas property sensor within the housing and positioned proximally from the acoustic receiver, the first gas property comprising at least one of a flow sensor, a pressure sensor, a humidity sensor, or a temperature sensor.

### CROSS-REFERENCE TO RELATED APPLICATIONS

This application claims the benefit of U.S. Provisional Application No. 63/376,009 filed Sep. 19, 2022, entitled “Acoustic Sensor Device with Integrated Gas Property Sensors,” which is incorporated herein by reference in its entirety.

### BACKGROUND

Medical ventilators offer critical support for patients who require ongoing breathing assistance. An endotracheal tube (ETT) is often used in conjunction with a ventilator, as part of a patient breathing circuit. The ETT is placed, via intubation, through the patient's mouth into the trachea, bypassing the upper airway. A number of sensors may be used with the ETT to ensure proper positioning within the trachea, and to ensure that the patient is receiving adequate ventilation. These sensors add weight and bulk to the ETT and add to the overall length of the breathing circuit, which may affect ventilatory efficacy. It is with respect to this general technical environment that aspects of the present technology disclosed herein have been contemplated.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In an aspect, the technology relates to an acoustic sensor device for connection to a tracheal tube. The acoustic sensor device includes a proximal end connectable to a breathing circuit to receive breathing gases; a distal end connectable to the tracheal tube; a housing, between the proximal end and the distal end, defining a lumen through which the breathing gases flow; an acoustic generator within the housing and positioned to emit acoustic pulses into the lumen; an acoustic receiver within the housing and positioned distally from the acoustic generator; and a first gas property sensor within the housing and positioned proximally from the acoustic receiver, the first gas property comprising at least one of a flow sensor, a pressure sensor, a humidity sensor, or a temperature sensor.

In an example, the housing defines a speaker cavity and the acoustic generator and the first gas property sensor are disposed within the speaker cavity. In another example, the acoustic sensor device further includes a second gas property sensor positioned distally from the acoustic receiver. In a further example, the first gas property sensor and the second gas property sensor are recessed into a wall of the lumen. In yet another example, the acoustic sensor device further includes a sampling channel defined by the housing, the sampling channel including an opening to the lumen. In still another example, the acoustic sensor device further includes an injection channel defined by the housing, the injection channel; and an injection nozzle coupled to the injection channel.

In another aspect, the technology relates to a method for capturing measurement data from an acoustic sensor device coupled to a tracheal tube. The method includes emitting, from an acoustic generator of the acoustic sensor device, an acoustic pulse into a lumen of the acoustic sensor device; detecting, by an acoustic receiver of the acoustic sensor device, the acoustic pulse; and based on the detected acoustic pulse, determining a position of the tracheal tube and a carbon dioxide concentration of breathing gases flowing through the lumen of the acoustic sensor device.

In an example, the method further includes receiving, from one or more sensors of the acoustic sensor device, temperature and humidity data for the breathing gases; and determining the carbon dioxide concentration is further based on the temperature and humidity data. In another example, the method further includes receiving, from a sensor of the acoustic device or a ventilator, flow rate data for breathing gases flowing through the lumen of the acoustic sensor; and wherein determining the carbon dioxide concentration is further based on the flow rate data. In still another example, the method further includes based on data from the acoustic receiver, determining breath phase data; and based on the breath phase data and the determined carbon dioxide concentration, determining an end-tidal carbon dioxide value. In still yet another example, the method further includes determining a speed of the acoustic pulse based on a transit time of the acoustic pulse between the acoustic receiver and another acoustic receiver of the acoustic sensor device; and wherein the wherein determining the carbon dioxide concentration is further based on the speed of the acoustic pulse.

In another aspect, the technology relates to a system for detecting breathing gas properties and tracheal tube positioning. The system includes an acoustic sensor device and a monitor communicatively coupled to the acoustic sensor device. The acoustic sensor device includes a proximal end connectable to a breathing circuit to receive breathing gases; a distal end connectable to the tracheal tube; a housing, between the proximal end and the distal end, defining a lumen through which the breathing gases flow; an acoustic generator within the housing and positioned to emit acoustic pulses into the lumen; a first acoustic receiver and a second acoustic receiver within the housing and positioned distally from the acoustic generator; a first gas property sensor within the housing and positioned proximally from the first acoustic receiver and the second acoustic receiver, the first gas property comprising at least one of a flow sensor, a pressure sensor, a humidity sensor, or a temperature sensor; and a second gas property sensor within the housing and positioned proximally from the first acoustic receiver and the second acoustic receiver, the second gas property comprising at least one of a flow sensor, a pressure sensor, a humidity sensor, or a temperature sensor. The monitor includes a display; a processor; and memory storing instructions that when executed by the processor cause the monitor to perform operations. The operations include receiving gas property sensor data from the first gas property sensor and the second gas property sensor; receiving acoustic data captured by the first acoustic receiver and the second acoustic receiver; displaying, on the display, gas property values based on the received gas property sensor data; and displaying, on the display, tracheal tube position based on the received acoustic data.

In an example, the acoustic sensor device further comprises a sampling channel defined by the housing, the sampling channel including an opening to the lumen; and the system further comprises a sampling tube pneumatically coupled to the monitor and the sampling channel. In another example, the monitor further comprises a capnography analyzer positioned to receive gas sampled through the sampling tube; and the operations further include analyzing the sampled gases to determine a carbon dioxide concentration; and displaying, on the display, the carbon dioxide concentration. In still another example, the acoustic sensor device further comprises an injection channel defined by the housing, the injection channel; and an injection nozzle coupled to the injection channel.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts an example medical ventilation system.

FIG. 2A depicts a cross-sectional view of an example acoustic sensor package.

FIG. 2B depicts a cross-sectional view of an example acoustic sensor package with a distal gas-sample measurement port.

FIG. 2C depicts a cross-sectional view of an example acoustic sensor package with a proximal gas-sample measurement port.

FIG. 2D depicts a cross-sectional view of an example acoustic sensor package with a nebulizer injection port.

FIG. 3 depicts an example method for capturing measurement data from an acoustic sensor package.

FIG. 4 depicts an example method for performing capnometry using acoustic sound speed data captured from an acoustic sensor package.

### DETAILED DESCRIPTION

An endotracheal tube (ETT) is a type of breathing tube that is placed through the nose or mouth of a patient and into the trachea. The proximal end of the tube remains outside the patient and is typically connected to a mechanical ventilator to form a patient breathing circuit. The ETT helps maintain patency of the airway, permits positive pressure ventilation; seals off the digestive tract from the trachea (thereby preventing inspiration of forced air into the stomach), and provides a means for supplying oxygen, anesthesia, or other breathing gases to a patient requiring respiratory support. An ETT is placed in the patient's airway by a clinician through a process called intubation. Once placed, the ETT may be affixed to the patient by medical tape, elastic harness, or other method, in order to help the distal end of the ETT remain in position in the trachea.

Despite action by a clinician to help keep an ETT in place in the trachea, the position of the ETT may drift over time, such that the distal tip of the ETT may relocate to a portion of the patient's anatomy that may reduce ventilatory efficacy or present a health or safety risk. For example, the ETT may drift inferiorly, towards or into the left or right bronchi, or into deeper branches of the bronchial tree. In other examples, the ETT may drift superiorly, towards or above the larynx. In examples where the ETT drifts superiorly, the dislocation of the ETT may be so significant that a clinician is required to perform an extubation, followed by a re-intubation, in order to position the ETT correctly. In similar examples, the ETT may be completely expelled by the patient, requiring re-intubation. This drift in ETT position may be especially problematic for pediatric patients, such as neonatal patients, preterm infants, or infants requiring intensive care, such as in a neonatal intensive care unit (NICU). Unplanned extubations are a sadly common adverse event affecting mechanically ventilated NICU patients (impacting nearly 1 in 5 patients) and may result in injury and/or significant increases in hospital costs and hospital stays.

One approach for monitoring the position of the proximal tip of the ETT is to use an acoustic reflectometry device, such as the device described in U.S. Pat. No. 10,668,240, titled “Acoustical Guidance and Monitoring System,” (hereinafter “the '240 patent”) which is incorporated herein by reference in its entirety. Briefly, the acoustic reflectometry device is connected to the proximal end of the ETT, between the ETT and tubing associated with a ventilator. The device uses a sound pulse generator to transmit acoustic pulses from the proximal tip of the ETT towards the distal tip. These pulses interact with the distal tip of the tube and the patient's anatomy, and are reflected back to the device, which also houses two or more acoustic receivers. The received acoustic reflections are processed and analyzed to determine positional drift of the ETT distal tip relative to a baseline position. The received reflections are also analyzed to determine whether the distal tip of the ETT has entered a larger or smaller diameter respiratory passageway, and to locate and characterize ETT blockages for suction therapy. The position and blockage information derived from the acoustic analysis is displayed on a monitor associated with the device. A clinician may then address positional shifts or blockages in the ETT to help prevent unplanned extubation.

In addition to the acoustic reflectometry device, a number of sensors and/or other medical devices may be connected to the ETT or other related parts of the patient breathing circuit. For example, external humidity, temperature, flow, or pressure sensors may be connected to the breathing circuit, in addition to any similar sensors that may be present in the ventilator or other associated medical equipment. Humidity and temperature sensors may be used to verify the quality of the air being supplied to the patient. For instance, air that is too dry, moist, or that is too hot/cold may lead to patient discomfort and/or a range of secondary health complications. Pressure and flow sensors may be used to monitor the movement of gases and gas pressurization within the breathing circuit.

The breathing circuit may also include sensors for analyzing the content of breathing gases. For example, sensors for measuring the oxygen (O₂) concentration of supplied breathing gases may be used and may be located within the ventilator itself. The concentration of carbon dioxide (CO₂) expelled by the patient during exhalation is also useful for assessing ventilatory status and the overall respiratory function of a patient. The measurement of CO₂ concentration is referred to as capnometry, and one metric output from this measurement is end-tidal CO₂ (EtCO₂). EtCO₂ is considered by some to be the fastest indicator of ventilatory compromise and thus may be useful to monitor.

While these sensors, combined with the acoustic reflectometry system, provide critical information to clinicians, the presence of multiple sensors in the breathing circuit may itself introduce complications for the patient. First, the combination of sensors, along with any associated cabling, tubing, or connection mechanisms, may add considerable bulk and weight to the ETT and/or tubing associated with the breathing circuit. For neonatal patients, the diameter of the ETT may be as little as a couple millimeters, while the size of sensors and associated cabling and tubing may be substantially larger. This size disparity may result in a breathing circuit that is physically difficult to manage in relation to such a small patient, which may lead to extubations or other complications.

Second, the wide array of sensors and associated cabling and tubing may complicate the logistics of patient care and management. For example, a patient in an intensive care setting (e.g., a NICU) may already be connected to a wide array of health sensors, medical devices, or other life support systems. Every sensor and system connected to a patient introduces its own set of risks and is another element that must be actively observed and managed by clinicians. Even though these sensors and systems may provide critical information and healthcare functionality, their presence may increase the overall healthcare burden when provided as disparate sensors and systems. Finally, the combination of a multitude of separate sensors within the breathing circuit introduces a volume of additional air that does not participate in gas exchange within the patient. This additional volume may be referred to as “dead space.” As an example, a mainstream capnometry measurement connection may add up to several inches to the overall length of the patient breathing circuit, thereby adding substantial dead space to the breathing circuit. As the ratio of the patient's tidal volume to the total dead space decreases, the ability to effectively ventilate the patient becomes increasingly challenging. Reducing the amount dead space in the breathing circuit, especially for neonatal patients where the total tidal volume of breathing gases is particularly small, facilitates ventilation in these patients.

The present technology, among other things, addresses the drawbacks described above by integrating an array of breathing gas sensors and other features into the packaging of an acoustic reflectometry device. Incorporating such breathing gas sensors into acoustic reflectometry devices present a special challenge to sensor integration. Acoustic reflectometry systems are sensitive to mechanical or spatial discontinuities in the vicinity of acoustic receivers associated with the device because these discontinuities can affect the transmitted and reflected acoustic pulses. Thus, placement of additional sensors or other features must be carefully considered and/or accounted for by the acoustic reflectometry system in order to avoid negatively affecting acoustic performance.

The discussion below presents systems and methods associated with the integration of breathing gas sensors such as temperature, humidity, flow, pressure, or other sensors, into an acoustic reflectometry device, along with one or more tubing connection ports. The tubing connection ports may provide a means for sampling and measuring breathing gases from the patient breathing circuit, or may provide a means for delivering inhalable medication, anesthesia, or other therapeutics to a patient. As described herein, the combination of breathing gas sensors, tubing connection ports, and acoustic reflectometry measurements in a single sensor package may significantly reduce the overall bulk and weight of the patient breathing circuit and may significantly reduce the dead space associated with each of these features, as compared to how they may be implemented in current practice. The consolidation of several sensing systems and breathing gas measurement capabilities into a single packaged device may also reduce the burden of care for clinicians, while providing critical information related to breathing gas characteristics, airway patency, or patient respiratory status in a single system. Further details are now provided by way of discussion of the accompanying drawings.

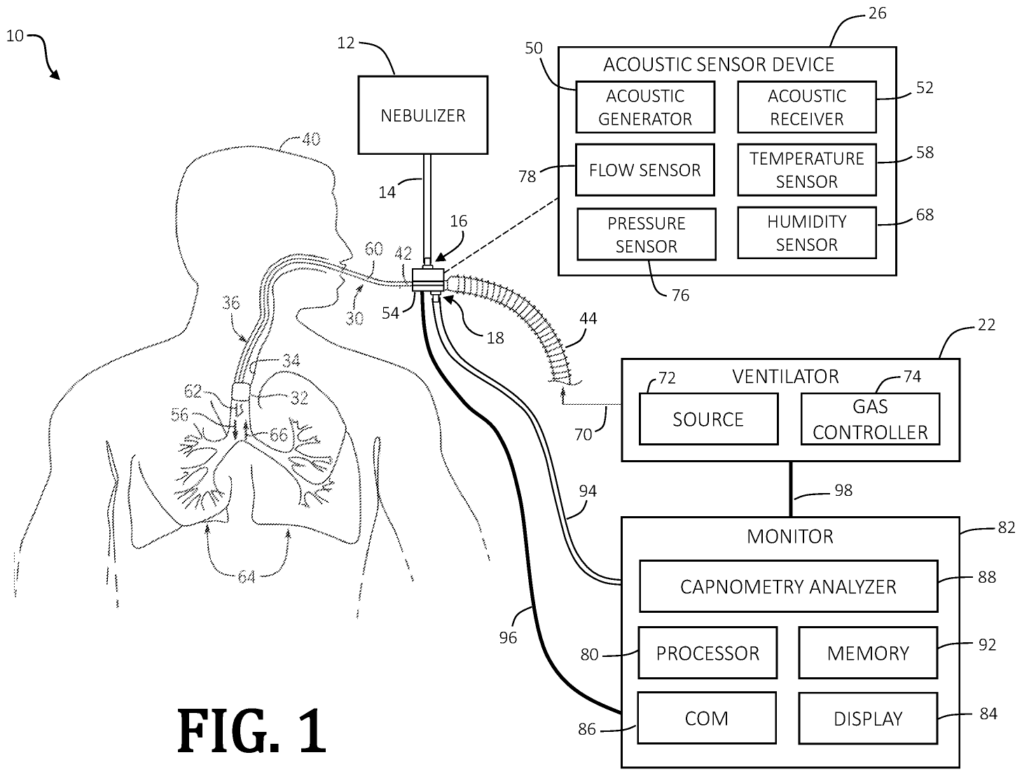

FIG. 1 depicts an example of an airway management system or medical ventilation system 10 that includes a ventilator 22, a monitor 82, an acoustic sensor package or device 26, and a tracheal tube 30. The tracheal tube 30 is illustrated as an endotracheal tube, which has an inflatable balloon cuff 32 that may be inflated to form a seal against walls 34 of a trachea 36 of a patient 40. However, the tracheal tube 30 may alternatively be uncuffed. The airway management system 10 may be used in conjunction with any other suitable types of tracheal tubes or medical devices. As examples, the airway management system 10 may be utilized with an endotracheal tube, an endobronchial tube, a tracheostomy tube, an introducer, an endoscope, a bougie, a circuit, an airway accessory, a connector, an adapter, a filter, a humidifier, nasal cannula, or a supraglottic mask/tube.

The system 10 includes devices that facilitate positive pressure ventilation of the patient 40, such as the ventilator 22, which provides mechanical ventilation to the patient 40. For example, the ventilator 22 may provide a gas mixture 70 (e.g., from a source 72 of the gas mixture 70) through the acoustic sensor package 26, through the tracheal tube 30, and to lungs 64 of the patient 40, thereby mechanically actuating rest, inspiration, and expiration phases of breathing cycles of the patient 40. The gas mixture 70 may be referred to herein as breathing gases. In some examples, the ventilator 22 includes a gas mixture controller 74 that provides control instructions to cause the ventilator 22 to continuously or intermittently adjust a pressure and/or a composition of the gas mixture 70 provided from the source 72 and to the patient 40. For example, the gas mixture controller 74 may cause the ventilator 22 to direct air, oxygen, or another suitable gas mixture from the source 72 to the lungs 64 of the patient 40.

As illustrated, the acoustic sensor package 26 of the airway management system 10 is coupled to an external or proximal end 42 of the tracheal tube 30. In the illustrated example, the acoustic sensor package 26 may operate as, or be, an adapter that facilitates coupling of the tracheal tube 30 to a patient circuit 44 or hose/tube coupled to the ventilator 22. Other arrangements are also contemplated, such as an acoustic sensor package 26 that is disposed on the tracheal tube 30 or on other components of the breathing circuit.

The acoustic sensor package 26 includes at least one acoustic generator 50 and at least one acoustic receiver 52 disposed within an adapter housing 54. The acoustic generator 50 is oriented to direct incident sound energy 56 (e.g., acoustic pulses, sound, acoustic energy) into the lumen 60 of the tracheal tube 30, which guides the sound energy 56 out of an internal or distal end 62 of the tracheal tube 30 and toward airways of lungs 64 of the patient 40. Further, the acoustic receiver 52 detects reflected sound energy 66, or echoes of the sound energy 56, back from different positions along the endotracheal tube/and or the airways of the patient. For instance, the emitted acoustic pulses may reflect from items such as obstructions in the tracheal tube, the tip of the tracheal tube, and the airways of the lungs 64. Accordingly, the acoustic sensor package 26 facilitates acoustic reflectometry techniques that analyze sound pressure waveforms for airway acoustic echoes indicative of airway size. That is, the acoustic generator 50 and the acoustic receiver 52 cooperate to provide sensor signals indicative of a sound pressure waveform having an airway acoustic echo, which the monitor 82 may analyze to determine, among other things, an airway size of the trachea around, or distally located from, the tip of the tracheal tube.

The acoustic generator 50 in some examples is a speaker or a miniature speaker. However, the acoustic generator 50 may additionally or alternatively include any suitable loudspeakers, buzzers, horns, sounders, and so forth that rely on moving coil, electrostatic, isodynamic, or piezo-electric techniques. Additionally, the acoustic receiver 52 may be a microphone, microphone array, or other sound pressure sensors, in some examples. When implemented as a microphone array, the acoustic receiver 52 and/or the monitor 82 discussed below may be designed to sense the direction from which sound energy is emitted and received and therefore isolate or filter out any interfering sound energy that is not a reflection of the emitted sound energy 56, provided by the acoustic generator 50, arising from the trachea tube 30 and airways of the lungs 64. Further, when implemented as a microphone array, the acoustic receiver 52 may be designed to determine the speed of travel or propagation (sound speed) of the emitted or reflected acoustic pulses in the region between sound sensing elements within the microphone array, or in the region between the acoustic generator 50 and one or more of the sound sensing elements. Other suitably paired components that respectively generate suitable sound energy and receive echoes or reflection of the sound energy may be used in the acoustic sensor package 26. Additional details regarding acoustic reflectometry are described in U.S. Pat. No. 9,707,363, titled “System and Method for Use of Acoustic Reflectometry Information in Ventilation Devices,” which is incorporated herein by reference in its entirety, and the '240 patent, referenced above. For instance, example components for the acoustic receiver(s) 52 and the acoustic generator 50 are described in the '240 patent.

In some examples, the acoustic sensor package 26 may not include an acoustic generator 50, or the acoustic generator 50 may be permanently or intermittently left unpowered. In such examples, one or more sound-sensitive elements of acoustic receiver 52 may acquire sounds generated naturally by the body of the patient 40. Analysis of these sounds may provide clinicians with relevant data or information about the health of the patient 40. For example, analysis of naturally occurring, internal body sounds may indicate the presence or onset of congestion, wheezing, pneumonia, and/or other health condition.

In other examples, when no acoustic pulses are coupled/directed into the lumen 60 by an acoustic generator 50, the acoustic receiver 52 may detect transmitted and/or reflected acoustic energy generated by other sources. For example, the ventilator 22 may inherently generate acoustic energy that couples into the lumen 60 of the tracheal tube 30, and may be suitable for performing some level of acoustic reflectometry, as described above. In some examples, other acoustic sources may provide suitable acoustic energy for performing acoustic reflectometry, such as sources that may be coupled to the patient circuit 44. Acquisition and analysis of acoustic energy provided by sources other than the acoustic generator 50 may include the use of specific filtering and/or data processing that differs from configurations where the acoustic generator 50 is the source of acoustic energy.

The acoustic sensor package 26 may include any combination of a plurality of sensors in addition to the acoustic sensors, such as a flow sensor 78, temperature sensor 58, pressure sensor 76, or humidity sensor 68. In examples, the flow sensor 78 may be a mass flow sensor, which measures the transfer of thermal energy to determine flow. For instance, the flow sensor 78 may be a mass flow sensor packaged in an integrated circuit (IC) or may be a “hot wire” type of design. In other examples, the flow sensor 78 may be mechanical based, where the gas flow is determined by measurement of moving parts. For instance, flow may be determined by rotation of a sensor element, such as a paddle wheel or propeller, or flow may be determined by movement of some other element or mechanism. The flow sensor 78 may include sensing mechanisms such as infrared (IR), ultrasound, electromagnetic, capacitive, inductive, or other type of sensing mechanism. In examples where acoustic receiver 52 is implemented as a microphone array, the flow may be estimated from correlation of received acoustic signals, such as from correlation of acoustic turbulence amplitude.

The temperature sensor 58 may be any of a wide variety of possible temperature sensing solutions. For instance, the temperature sensor 58 may be based on a thermistor, thermocouple, thermopile, resistance temperature detector (RTD), semiconductor, IR, bimetallic element, liquid expansion element, or any other type of temperature responsive element. The temperature measurements provided by the temperature sensor 58 allow the temperature of supplied breathing gases to be observed at a measurement point closer to the patient than other potential temperature sensors in the patient breathing circuit. A clinician may use the measurements to adjust the temperature setpoint on the ventilator 22 and/or a humidifier (not shown). As described below, the temperature measurements may also be used in conjunction with other measurements, such as with capnography measurements, or for compensation for other measurements performed within the acoustic sensor package 26.

The pressure sensor 76 may be any of a variety of possible pressure sensing solutions. In some examples, the pressure sensor 76 may be based on a strain gauge, or a resistive, capacitive, inductive, MEMS, or other type of sensing element. The pressure sensor 76 may be based on a deflection-type mechanism, such as a diaphragm or other deflectable element, or the pressure sensor 76 may be based on another type of pressure sensitive mechanism. The pressure sensor 76 may provide pressure data to the monitor 82 independently of any pressure sensors that may be present in the ventilator 22 or in other portions of the patient breathing circuit.

In some examples, a separate pressure sensor 76 may be omitted and the pressure measurements of the breathing gases may be measured by the acoustic receiver(s) 52 of the acoustic sensor package. For instance, low frequency pressure changes may be detected by the acoustic receiver(s) 52. As an example, the acoustic receiver 52 may include a piezo-electric film and low-frequency deflection of the film may be attributed to pressure changes of the breathing gases. The pressure changes may also be used to determine breathing phases, such as an inspiratory phase and an expiratory phase. Additional details regarding pressure measurements are provided in the discussion relating to FIG. 12 in the '240 patent.

The humidity sensor 68 provides a means of measuring the humidity of supplied gases to the patient. In some examples, the humidity sensor may be a relative humidity sensor, such as one based on capacitive or resistive sensing elements. In other examples, the humidity sensor 68 may be an absolute humidity sensor, such as a thermal conductivity sensor. In still other examples, the humidity sensor 68 may comprise other types of humidity sensitive measurement elements and/or mechanisms. A clinician may use the humidity measurements to adjust the humidity setpoint of the ventilator 22 or of an external humidifier (not shown) associated with the patient breathing circuit.

Within the acoustic sensor package 26, the temperature sensor 58, humidity sensor 68, pressure sensor 76, and/or flow sensor 78 may each include individual sensing elements (such as the types of sensing elements or mechanisms described above). Any support circuitry for the individual sensing elements may also be incorporated with the temperature sensor 58, humidity sensor 68, pressure sensor 76, and/or flow sensor 78. For instance, the support circuitry may include active or passive electronic components such as resistors, capacitors, inductors, semiconductor devices (e.g., diodes), power regulation ICs, or other types of electronic components. In still other examples, the sensors may each include an IC communicatively connected to a sensing element, or the sensors may each include a fully integrated sensing module, which may include an IC or ICs, active or passive support circuitry, and/or the sensing elements. The sensors may include any combination of the above. For example, one or more of the sensors may include an individual sensing element, and the remaining sensors may comprise a more fully integrated sensing module, which is consolidated in an IC. In other examples, the sensors may all comprise fully integrated sensing modules.

An example of a more fully integrated sensing module may be one in which analog signals transduced by a sensing element or mechanism are conditioned or processed, and converted to a digital format, such as by an analog-to-digital converter (ADC). For instance, the temperature sensor 58 may include a temperature transducer (e.g., a thermistor), a bridge circuit, filter, amplifier, ADC, and back-end digital processing circuitry. The digital temperature data may then be transmitted to the monitor 82 and visualized by the clinician on display 84, without the need for analog signal conditioning within the monitor 82. As an alternative example, the temperature sensor 58 may include only a temperature transducer (e.g., a thermistor), with the remaining elements (e.g., bridge circuit, filter, amplifier, ADC, and/or back-end digital processing circuitry) existing within the monitor 82. In examples where the sensors 58, 68, 76, or 78 include only basic sensing elements, or minimal analog signal conditioning, further analog or digital signal processing may take place in the monitor 82.

The acoustic sensor package 26 is not limited to the types and number of sensors illustrated in FIG. 1. A greater or fewer number of sensors may be included in the acoustic sensor package 26, and different types of sensors may be included to observe different types of clinically relevant measurands. Further, it should be understood that the sensors may be integrated into the acoustic sensor package 26 as separate elements, with or without supporting circuitry, as illustrated in FIG. 1, or two or more of the sensors may be combined into a common package, such as an IC, to further consolidate the sensors into a smaller volume within the acoustic sensor package 26.

In addition to the acoustic sensor package 26, the adapter housing 54 may include one or more ports, such as a nebulizer port 16 and/or a sampling port 18. The nebulizer port 16 may allow for the connection of a nebulizer tube 14, which provides a conduit for nebulizer 12 to impart supplemental therapeutics into the breathing gases supplied to the patient 40. For example, the nebulizer 12 may allow clinicians to provide inhalable medicine, therapeutics, anesthesia, or other type of inhalation-based therapy to the patient 40 via the breathing gases supplied within the patient circuit 44. The adapter housing may also provide a sampling port 18 for capturing samples of breathing gases inhaled and exhaled by the patient 40. For example, a sampling tube 94 may be connected to the sampling port 18, thereby providing a conduit for the monitor 82 to draw or siphon gas samples from the inhaled and exhaled breathing gases. Accordingly, the sampling tube 94 may be pneumatically coupled to the monitor 82. As a further example, the monitor 82 may provide a capnometry analyzer 88 for determining CO₂ concentration based on the sampled gases. In such an example, the sample port 18 may serve as a mainstream or sidestream CO₂ monitoring port for EtCO₂ measurements.

The adapter housing 54 may include both nebulizer port 16 and sampling port 18, one port or the other, or neither port, depending on the configuration of the adapter housing 54 and on clinical need. The inclusion of one port or the other, or both the nebulizer port 16 and the sidestream port 18, may greatly reduce dead space within the patient circuit 44. In the example given above, the use of sampling port 18 as a sidestream capnographic monitor may eliminate the need for a mainstream capnographic monitor within the patient circuit 44. A separate mainstream capnographic monitor adds significant dead space within the patient circuit 44 and adds significant bulk to patient circuit 44.

The acoustic sensor package 26 may be communicatively coupled to the monitor 82 via interface cable 96. The interface cable 96 provides a means for sensor measurement data to be transmitted from the acoustic sensor package 26 to the monitor 82. Elements within the acoustic sensor package 26 may transmit other types of data to the monitor 82 by way of the interface cable 96. For example, elements within the acoustic sensor package 26 may transmit status, information related to error conditions, device settings, configuration information, or other types of information. Additionally or alternatively, the monitor 82 may transmit information to the acoustic sensor package 26 via interface cable 96. For instance, the monitor 82 may transmit sensor operating parameters, settings, configuration information, data, variables, firmware, executable code, or other information or data associated with acoustic sensor package 26 operation. The interface cable 96 may also provide a means for the monitor 82 to provide electrical power to the acoustic sensor package 26.

The interface cable 96 may transmit any combination of analog or digital signals between the acoustic sensor package 26 and the monitor 82. The interface cable 96 may be a single cable including two or more conductors or the interface cable 96 may comprise a bundled set of cables or wires. For example, the acoustic generator 50, acoustic receiver 52, and the sensors 58, 68, 76, and 78 may have each have a dedicated cable or wire(s), which may be bundled as interface cable 96. In other examples, the acoustic generator 50, acoustic receiver 52, and the sensors 58, 68, 76, and 78 may be bundled in any combination of a set of cables. For instance, the acoustic elements (generator 50 and receiver 52) of the acoustic sensor package 26 may interface with the monitor 82 by way of a first cable bundled within interface cable 96, and the sensors 58, 68, 76, and 78 may interface with the monitor 82 by way of a second cable bundled within interface cable 96. Accordingly, in examples where the interface cable 96 is a single cable, the interface cable 96 may comprise a single connector on each end (not depicted in FIG. 1 for visual clarity), or in examples where the interface cable 96 comprises a set of bundled cables, a corresponding number of connectors may be included. In other examples, interface cable 96 may comprise a hardwired connection on either end where no connectors are necessary.

The monitor 82 may include a communication connection(s) 86 (e.g., input/output ports, communication circuitry, analog interface circuitry), user-selectable buttons, a memory 92, and processing circuitry, such as the processor 80, that are communicatively coupled to one another. The communication connection(s) 86 may receive data from, and/or transmit data to, the acoustic sensor package 26 via interface cable 96. The communication connection(s) 86 may interface with the acoustic sensor package 26 in any of a wide variety of digital communications protocols, such as Serial Peripheral Interface (SPI), Inter-Integrated Circuit (I2C), Universal Asynchronous Receiver Transmitter (UART), or other established or custom digital protocols. In some examples, the communication connection(s) 86 may receive analog signals from sensors within the acoustic sensor package 26, such as analog signals produced directly by sensing elements within the acoustic sensor package 26. The communication connection(s) 86 may provide signal level translation, bias voltages or currents, or other signals or functions needed to interface with the acoustic sensor package 26. In other examples, the communication connection(s) 86 may communicate with the acoustic sensor package 26 via wireless protocols, such as WiFi, Bluetooth, or other established or custom wireless transceiver protocol. In some examples the communication connection(s) 86 may also provide electrical power to the acoustic sensor package 26. In some instances, the communication connection(s) 86 may transmit or receive additional signals to or from the acoustic sensor package 26, such as clock or timing signals, status signals, or other types of signals used in operation.

Processing circuitry, such as processor 80, may include any combination of digital and analog circuitry needed for processing signals received from, or transmitted to, the acoustic sensor package 26. Examples of analog processing circuitry may include signal filters, amplifiers, bridge circuits, bias circuits, pulse generators, level translators, analog-to-digital converters (ADCs), or other types or combinations of analog circuitry. The processor 80 may include one or more general purpose processors, microprocessors, microcontrollers, digital signal processors (DSPs), or other programmable circuits. In examples, the processor 80 may include any combination of commercially available components, or custom or semi-custom integrated circuits, such as application specific integrated circuits (ASICs). The processor 80 may include elements needed for control or communication with the display 84, memory 92, communication connection(s) 86, and capnometry analyzer 88. The processor 80 may perform control, interface, communication, or other processing functions by executing instructions that are stored in the memory 92. The memory 92 may include RAM, ROM, electrically erasable programmable read-only memory (EEPROM), flash memory or other memory technology, CD-ROM, digital versatile disks (DVD) or other optical storage, magnetic cassettes, magnetic tape, magnetic disk storage or other magnetic storage devices, or other types of storage media.

The processor 80 may direct information to be displayed on the display 84. The information may include data collected from any of the sensors housed within the acoustic sensor package 26 and/or output from the capnometry analyzer 88. In other examples, the information to be displayed may include status or other operating information related to the monitor 82. In examples where the display 84 is a touch-sensitive screen (e.g., a capacitive touch-sensitive screen), the processor 80 may receive user inputs from the display 84 and respond accordingly. The display 84 may be any of an LED, LCD, OLED, or other display technology. In still other examples, the display 84 may include any type of visual indicator such as lamps, bulbs or strip light sources, or other types of discrete visual indicators.

The monitor 82 may also receive user input from user-selectable buttons or other mechanical or electrical input which may be provided to the user on the housing of the monitor 82. The user inputs may allow clinicians to select the sensor data to be displayed, select the format of the display, set alert conditions, set alert thresholds, respond to alerts, search menus, configure the monitor 82, configure the acoustic sensor package 26, configure the capnometry analyzer 88, retrieve stored settings, retrieve stored measurements, and/or provide other types of input to the monitor 82, among other functions.

In some examples, the monitor 82 includes a capnometry analyzer 88 for determining and displaying data related to CO₂ concentration. The capnometry analyzer 88 may include instrumentation for determining CO₂ concentration using samples of breathing gases drawn or siphoned through sampling tube 94. For example, the capnometry analyzer 88 may utilize an IR-based sensing mechanism for measuring the amount of CO₂ in the breathing gas samples. In other examples, the capnometry analyzer 88 may use other types of instrumentation for measuring CO₂ concentration directly from samples of the breathing gases. In such examples, the capnometry analyzer 88 and/or monitor 82 may include necessary elements or mechanisms to draw or siphon the required gas samples from the patient breathing circuit, such as pumps, tubing, tubing ports, couplings, motors, or other electrical or mechanical elements or mechanisms.

Additionally or alternatively, the capnometry analyzer 88 may determine CO₂ concentration using acoustic data received by the monitor 82 from the acoustic sensor package 26. For instance, the capnometry analyzer 88 may use sound speed to determine CO₂ concentration. As an example, during the exhalation portion of the breathing cycle, CO₂ is expelled by the patient. The speed of propagation of the acoustic pulses through the tracheal tube 30 and acoustic sensor package 26 is affected by the composition of gas, including the presence of CO₂, and changes in sound speed may be detected by the acoustic receiver 52. For example, when the acoustic sensor package 26 includes two acoustic receivers 52 offset by a set distance, the time an acoustic pulse takes to travel from the first acoustic receiver to the second acoustic receiver may be used to calculate the sound speed. In other examples, the time an acoustic pulse takes to travel from the acoustic generator 50 to a sound-sensitive element of the acoustic receiver 52 may be used to calculate sound speed. Acoustic signals transduced by the acoustic receiver 52 may be processed by analog and/or digital circuitry associated with any of the acoustic receiver 52, processor 80, and/or capnometry analyzer 88 to produce sound speed data that may be used in capnometry analysis. The capnometry analyzer 88 may output capnometry data in graphical or numeric form for presentation on the display 84.

Similar to the example provided above for capnometry, the sound generating and receiving elements of the acoustic sensor package 26 may be used to determine changes in concentration of other breathing gases. In examples, changes in O₂ concentration, the introduction of anesthetic gases, and/or other changes to breathing gas composition and concentration may be detectable as a change in pulse transit time between acoustic elements, such as between the acoustic generator 50 and a sound sensitive element of an acoustic receiver 52. The change in pulse transit time may then be used to determine sound speed between the acoustic elements, which may be correlated with changes in partial pressure or concentration of a particular gas.

As described below, using pulse transit time to determine changes in gas concentration may include the use of additional data or contextual information. In some examples, temperature and humidity measurements of the breathing gases may be included in the concentration analysis, as these variables are relevant to partial pressure and concentration. In other examples, contextual information may be included in the concentration analysis. For instance, the current phase of the breathing cycle (as may be provided by the ventilator 22) may be included, since levels of O₂ and CO₂ are expected to fluctuate during the breathing cycle. Specifically, O₂ concentration may be higher during inhalation and lower during exhalation, and conversely, CO₂ would be expected to be higher during exhalation and lower during inhalation. Thus, information on the phase of the current breathing cycle may inform the gas concentration analysis. Similarly, an indication of the delivery of anesthetic gases and/or the type of anesthetic gases delivered may inform the gas concentration analysis. As an example, an increase in O₂ concentration may increase the sound speed, whereas an increase in anesthesia gases may reduce the sound speed. The relative changes in sound speed may thus be used to estimate changes in partial pressures (or concentrations) of introduced gases, such as anesthesia gases. This gas concentration analysis may be performed by an element of the airway management system 10, such as by monitor 82.

As described, the monitor 82 receives and processes sensor data from the acoustic sensor package 26 via interface cable 96. The monitor 82 may receive the sensor data in a digital format or may receive analog signals from sensing elements or mechanisms associated with sensors 58, 68, 76, or 78. These received analog signals may require further signal conditioning by circuitry associated with the communication and interface connection(s) 86 or processor 80. Additionally or alternatively, the monitor 82 may be configured by the user, or by the monitor manufacturer, to perform digital signal processing, such as performing digital filtering or other statistical digital process. Based on how the monitor 82 is configured, the monitor 82 may display any combination of the data received from the acoustic sensor package 26, along with any data provided by the capnometry analyzer 88, such as diameter of the airway around the distal tip of tracheal tube 30; the location and size of blockages within the tracheal tube 30; other characteristics of the patient's airway (e.g., detection or identification of tracheal collapse); temperature of breathing gases within the acoustic sensor package 26; absolute or relative humidity of breathing gases; the amount and direction of breathing gas flow; relative, absolute, or gauge air pressure within the patient breathing circuit; EtCO₂; or other data provided by the acoustic sensor package 26 and/or capnometry analyzer 88.

Additionally or alternatively, the monitor 82 may display graphical representations of the data. For example, the monitor 82 may display a graphical representation of CO₂ concentration versus time, along with a numeric value for EtCO₂. The monitor 82 may display statistical information corresponding to data received from the acoustic sensor package 26 and/or capnometry analyzer 88. In examples, the monitor may display minimum values, maximum values, average values, mean, standard deviation, or any other statistical measure of sampled data, which may be computed by the processor 80. For example, the monitor 82 may store intervals of the measured data (collected over time) in memory 92, and the processor 80 may compute statistical measures for display. As a further example, the monitor 82 may store received temperature data collected over a user-specified interval (e.g., 5 minutes) in memory 92, and the processor 80 may calculate an average temperature over that interval based on the stored data. In other examples, the processor 80 may perform a running average over a user-specified interval, and continually display the average, or may display a graphical representation of the running average. In still other examples, the processor 80 may perform statistical calculations that may be provided by its programming on combinations of measurement data. The processor 80 may continually perform statistical calculations as new measurement data is received and stored, and the processor 80 may continually update the results of these calculations on the display 84, in either numeric or graphical form, or both. The monitor 82 may provide the capability for the user to configure the display or display format of any measured data and may provide the ability for the user to configure and display various statistical measures of the measured data.

In examples, the monitor 82 may provide visual and/or audio alerts to clinicians for conditions such as the distal tip of the tracheal tube 30 being mispositioned (e.g., too high or low) in the patient's airway, an extubation of the tracheal tube 30, a blockage detected in the tracheal tube 30, temperature of breathing gases out-of-range, pressure of breathing gases out-of-range, humidity of breathing gases out-of-range, flow of breathing gases out-of-range, EtCO₂ out-of-range, other indications of abnormal CO₂ concentration; or other conditions that may pose a risk to a patient for which the monitor 82 has corresponding data. The monitor 82 may allow alert conditions and/or thresholds to be configured by a user of the device. For example, a clinician may program and store thresholds for alert conditions using user-selectable buttons, other mechanical or electrical inputs, or a touch-sensitive display (such as through display 84) if these capabilities are provided by the monitor 82. The processor 80 may observe received data from the acoustic sensor package 26 and/or data from the capnometry analyzer 88 and detect conditions for which it has been configured to issue an alert. The processor 80 may access stored data, variables, thresholds, or other indicators stored in memory 92 that may be related to alert conditions.

In some examples, the monitor 82 may be communicatively coupled to the ventilator 22 via ventilator cable 98. The communication connection(s) 86 may be configured to transmit and/or receive information from the ventilator 22. The monitor 82 may provide measurement data to the ventilator 22, such as temperature, humidity, pressure, flow, CO₂ concentration, or other measurement data available to the monitor 82. The ventilator 22 may be configured to respond to the received data by automatically adjusting characteristics of the breathing gases supplied to the patient. For example, the ventilator 22 may automatically adjust operation of an associated humidifier (if so equipped) to increase or decrease the moisture content of the supplied breathing gases, based on the humidity level measured by the humidity sensor 68 within the acoustic sensor package 26. As an example, the ventilator 22 may use flow data received from the monitor 82 to detect airway collapse or other critical respiratory condition experienced by the patient. In still other examples, the ventilator 22 may execute an algorithm that combines any internal sensor data with any measurement data received from the monitor 82 to detect abnormal or adverse respiratory conditions or to automatically adjust various aspects of ventilatory output. In further examples, the ventilator 22 may provide a visual or audible alert of an abnormal, adverse, or out-of-range operating condition based at least in part on received measurement data from monitor 82. The monitor 82 may also provide data to other components, such as a humidifier. The humidifier may then increase or decrease the moisture content of the supplied breathing gases based on the humidity level measured by the humidity sensor 68 within the acoustic sensor package 26.

Additionally or alternatively, the ventilator 22 may provide information to the monitor 82 via ventilator cable 98. For example, the ventilator 22 may provide oxygen concentration data to the monitor 82 for use in capnometry analysis. In other examples, the ventilator 22 may also provide a variety of status or operational indications, or measurement data, to the monitor 82 for general-purpose use. In examples where the monitor 82 and ventilator 22 are not communicatively coupled (such as when ventilator cable 98 is not in use), a clinician may still use the data displayed on the monitor 82 to manually adjust any operating settings on the ventilator 22. For example, a clinician may adjust humidity or air temperature settings on the ventilator or associated equipment based on data displayed on the monitor 82.

While the monitor 82 is depicted as being separate from the ventilator in FIG. 1, in other examples, the functionality of the monitor may be integrated into the ventilator 22. For instance, the configuration, processing, and/or display capabilities discussed above with respect to the monitor 82 may be integrated into the ventilator 22. In such examples, the ventilator 22 may display the data associated with the acoustic sensor package 26 on a primary display of the ventilator 22. In other examples, the ventilator 22 may include a secondary display for displaying the data associated with the acoustic sensor package 26.

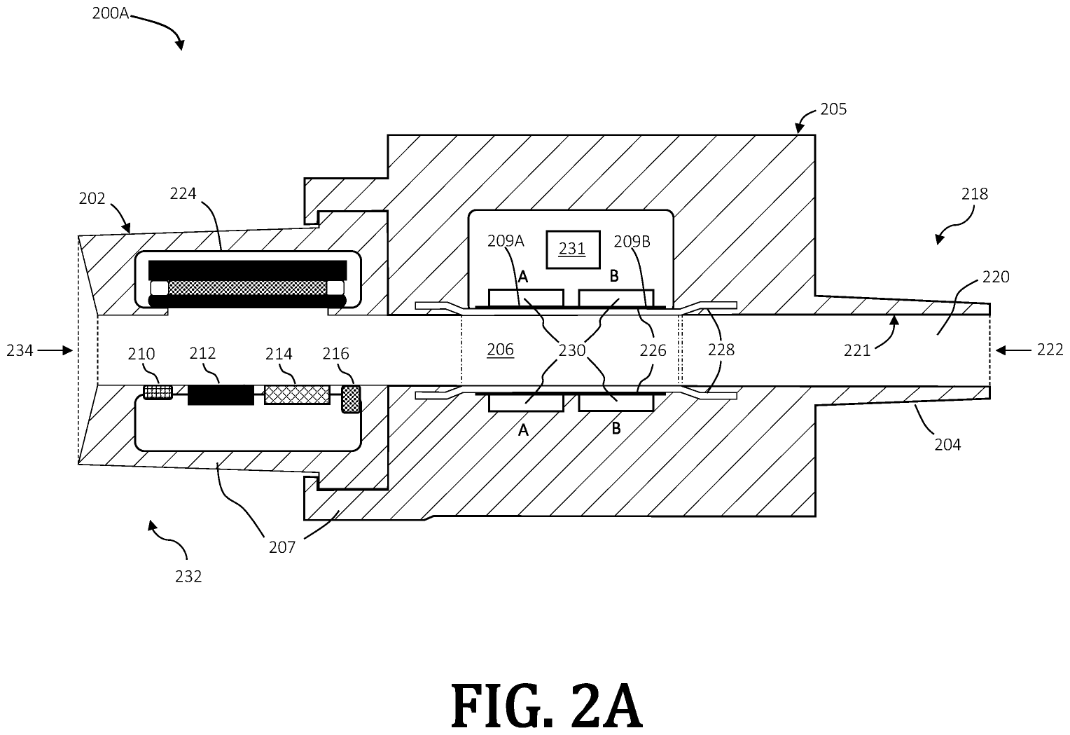

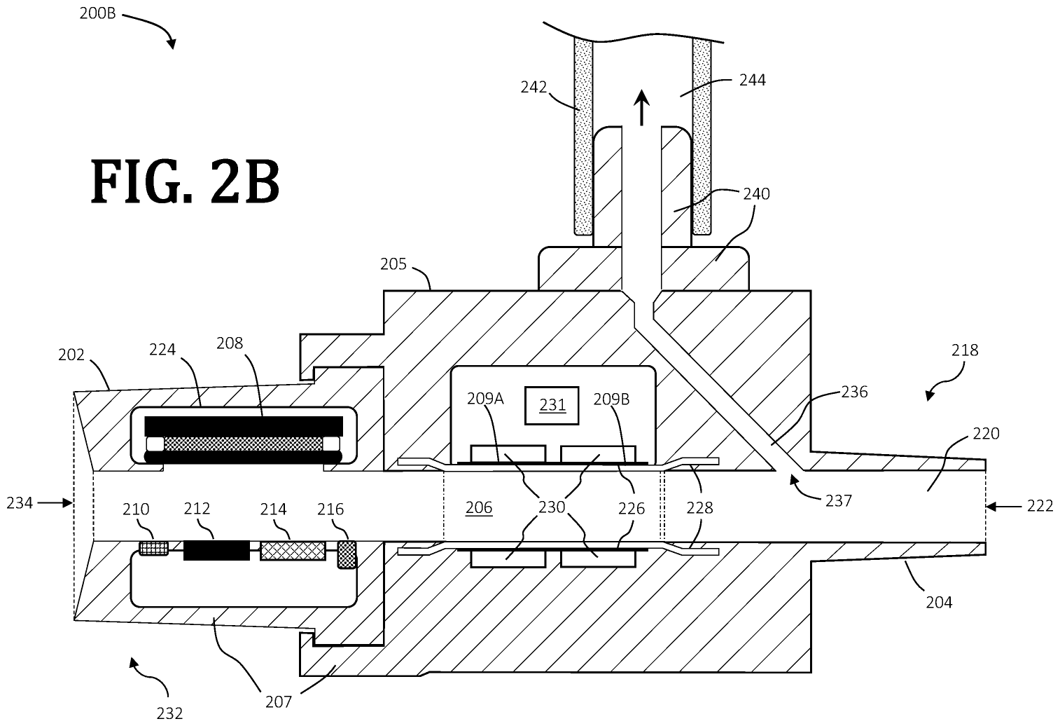

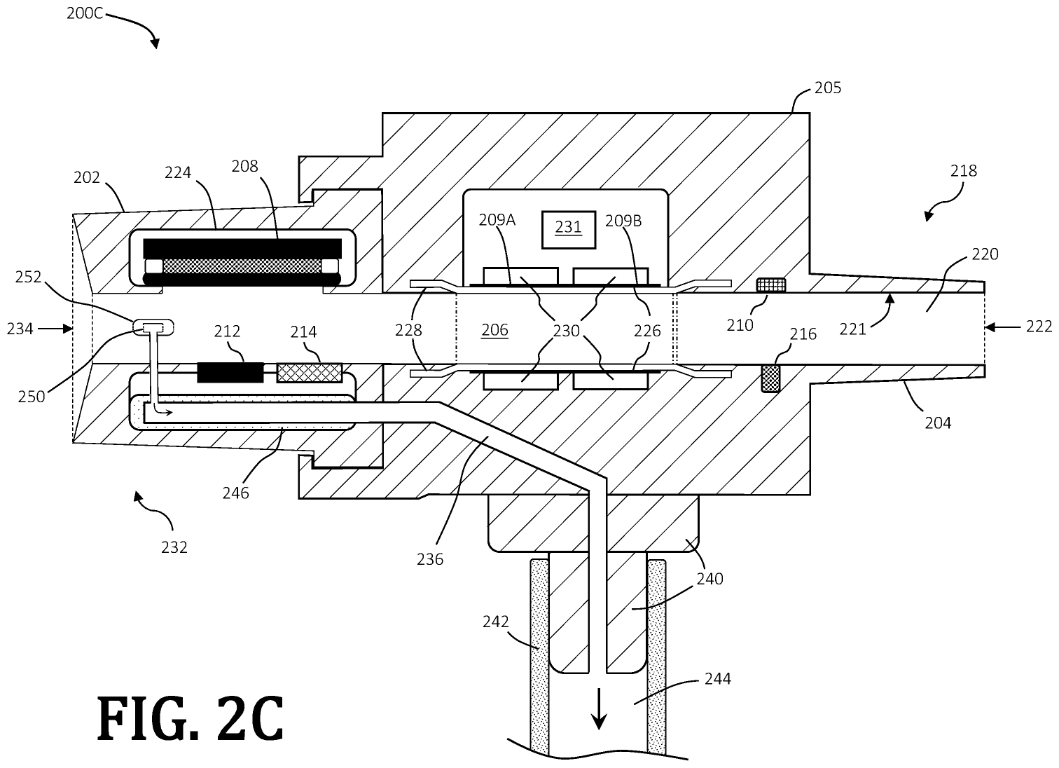

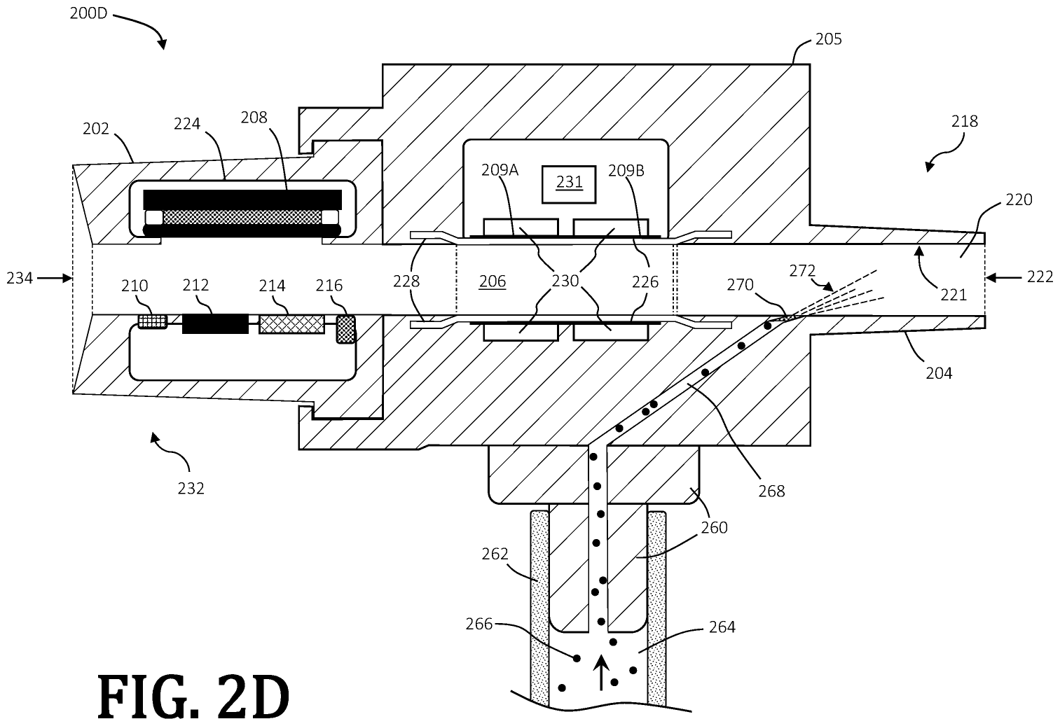

FIG. 2A depicts a cross-sectional view of an example acoustic sensor package 200A, which may represent the acoustic sensor package 26 described above. The sensor package 200A includes a proximal portion 232 and a distal portion 218. The distal portion 218 includes a nozzle 204 which may couple to an ETT (such as tracheal tube 30), with breathing gases flowing from the example acoustic sensor package 200A to the ETT through the distal opening 222. The proximal portion 232 may include a fitting 202 (e.g., a 15 mm fitting) for connecting tubing associated with a ventilator (such as ventilator 22 and patient circuit 44). Breathing gases are passed from tubing connected to the fitting 202 into an acoustic package lumen 220 through the proximal opening 234. The lumen 220 is an internal passageway defined by a housing 207 of acoustic sensor package 200A through which the breathing gases pass. Exhaled gases from the patient travel in the opposite direction (e.g., from the distal portion 218 to the proximal portion 232).

Disposed within the fitting 202 may be a speaker cavity 224, which houses a speaker 208 (a form of acoustic generator, such as acoustic generator 50). The speaker cavity 224 may wrap around the lumen 220. Gas property sensors 210, 212, 214, and 216 are incorporated into the example acoustic sensor package 200A, and one or more of the sensors 210, 212, 214, and 216 may be positioned within the speaker cavity 224. The sensors 210, 212, 214, and 216 may be any of a temperature sensor, humidity sensor, pressure sensor, and/or flow sensor, or may represent other types of sensors provided within the example acoustic sensor package 200A. For example, sensors may include a flow sensor 210, a pressure sensor 212, a humidity sensor 214, and a temperature sensor 216. As described, the sensors 210, 212, 214, and 216 may be individual sensing elements, or may be more fully integrated sensing modules, where the sensing elements are packaged with additional signal processing circuitry. The gas property sensors 210, 212, 214, and 216 transduce or generate gas property data.

The housing 207 of the example acoustic sensor package 200A also includes a sound-sensing section 205 coupled or attached to the fitting 202. The sound-sensing section 205 of the example acoustic sensor package 200A includes elements associated with detection and processing of the acoustic pulses generated by the speaker 208. In the example depicted, a sensor tube 228 provides a pliable substrate for coupling the acoustic pulses propagating in the acoustic package lumen 220 to the piezo-electric film 226. The piezo-electric film 226 functions as two sound-sensitive elements 209 (e.g., microphones) that transduce the received acoustic pulses into electric signals based on vibrations of the piezo-electric film 226. Along the length of the lumen 220 is a location A where the first sound-sensitive element 209A may be located, with a second sound-sensitive element 209B located more distally at location B The first sound-sensitive element 209A may be considered a part of a first acoustic receiver, and the second sound-sensitive element 209B may be considered a part of a second acoustic receiver. Foam sheets 230 may provide mechanical backing for improving the response of the sound-sensitive elements.

A printed circuit board (PCB) 231 may also be incorporated into the sound-sensing section 205. The PCB 231 may include electronics for generating signals transmitted as acoustic pulses by the speaker 208 into the acoustic package lumen 220, and for processing signals generated by the pulses received by the sound-sensitive elements within the piezo-electric film 226. For ease of visualization, interconnections between the PCB 231 and the speaker 208 are not depicted in FIG. 2A. The PCB 231 may also include interface electronics for transmitting data to, or receiving data from, the monitor 82 via interface cable 96. A connection mechanism for interface cable 96 (e.g., a connector or hardwire attachment) is also not depicted in FIG. 2A, for ease of visualization, but it should be understood that such a connection mechanism may be chosen from any of a wide variety of available connection options and may be implemented at locations with sufficient space within the acoustic sensor package 200A.

In operation, the speaker 208 transmits an acoustic pulse (or other form of emitted sound energy) into the lumen 220. The acoustic pulse travels distally into a sound sensor lumen 206, where the acoustic pulse first passes the first sound-sensitive element at location A, followed by the second sound-sensitive element at location B. The acoustic pulse continues to propagate distally (i.e., in a proximal-to-distal direction), into the ETT and into the airway of the patient. The acoustic pulse interacts with the patient's anatomy and is reflected back into the ETT (i.e., the reflected acoustic pulse now propagates in a distal-to-proximal direction), and subsequently propagates back into lumen 220. The reflected pulse first passes the second sound-sensitive element at location B, followed by the first sound-sensitive element at location A. The received pulse is transduced by the sound-sensitive elements and the corresponding signals processed by electronics on the PCB 231, with additional processing and display occurring at the monitor 82. The processed signals provide an indication of parameters associated with the distal end of the ETT tip, such as changes in location of the distal tip, diameter of the airway around or distally from distal tip, or blockages along the length of the ETT, among other parameters. The sound speed of the transmitted and/or reflected acoustic pulse may be calculated by determining the pulse transit time between the sound-sensitive elements at locations A and B, as described in more detail below. Additional acoustic pulses are transmitted on a recurring basis so that the ETT may be continually monitored.

In the example acoustic sensor package 200A, sensors 210, 212, 214, and 216 are located proximal to the sound-sensitive elements located within the piezo-electric film 226. Discontinuities within the lumen 220 and the downstream ETT may affect the transmitted and reflected acoustic pulses received by the sound-sensitive elements. As an example, a mechanical discontinuity in the lumen 220 located distally from the speaker 208 and/or sound sensing elements may impact the operation of the acoustic functions of the acoustic sensor package 200A. For instance, the discontinuity may cause an undesired reflection of the acoustic pulse and/or a loss of energy of the acoustic pulse that ultimately affects the position determinations based on the detected acoustic pulses. Accordingly, locating sensors 210, 212, 214, and 216 proximal to the sound-sensitive elements reduces the impact these sensors may have on the acoustic pulses, thereby reducing the probability of incorrect measurements based on the acoustic pulses.

If the discontinuity caused by a particular sensor, however, is sufficiently small and stable, any impact on the received acoustic signals may be accounted for by additional processing techniques and/or calibrations. As a result, for some sensors that can be integrated into a wall 221 of the lumen 220 in a substantially flush manner (e.g., minimal protrusion into the lumen 220) may be incorporated into positions that are distal from the speaker 208 and/or sound-sensing elements.

Depending on the particular sensor type, the sensors 210, 212, 214, or 216 may require direct access to the breathing gases flowing through the lumen 220 (e.g., to perform accurate measurements of the breathing gases within the lumen 220). In some examples, the sensors 210, 212, 214, or 216 may be recessed from the acoustic package lumen wall 221, while still maintaining access or exposure to the breathing gases in the lumen 220, to again minimize impact on the acoustic pulses. FIG. 2A illustrates an example where sensors 210, 212, 214, and 216 are mounted substantially flush with the acoustic package lumen wall 221. In other examples, one or more of the sensors may be embedded within the acoustic sensor package housing 207 but not have direct access to the breathing gases in the lumen 220. Nevertheless, such sensors may still measure some breathing gas characteristics. For instance, the housing 207 may be of a suitable thermal conductivity, such that a temperature sensor may still accurately measure the temperature of breathing gases while being embedded within the acoustic package sensor housing 207 (without direct access to the acoustic package lumen 220).

While the recessed or flush form of the sensors 210, 212, 214, or 216 may be more easily positioned at various locations within the lumen 220 and not have a significant effect on the acoustic functions, other forms of the sensors 210, 212, 214, or 216 may protrude more significantly into the lumen 220 and such sensors should generally be placed proximally from at least the sound-sensing elements to limit the affect on the acoustic functions. For example, instead of the flow sensor 210 being a recessed chip package, the flow sensor 210 may be a hot wire anemometer that uses a hot wire that extends across the lumen 220. As another example, the temperature sensor 216 may be a probe that protrudes into the lumen 220. Such protruding types of sensors may be placed proximate of the acoustic elements to limit interference.

FIG. 2B depicts a cross-sectional view of an example acoustic sensor package 200B, which includes a gas-sampling measurement port 240. A gas-sampling tube 242 is connected to the gas sample measurement port 240. In examples, the gas-sampling measurement port 240 may a Luer connection, any other type of standardized tubing connection, or a custom type of tubing connection. The opposite end of gas-sampling tube 242 may be connected to a monitor (such as monitor 82) or may be connected to another medical device suitable for performing measurements of sampled breathing gases. In some examples, the gas-sampling tube 242 may be connected to a ventilator (such as ventilator 22), which may be equipped to perform breathing gas measurements.

A sampling channel 236 is also defined by the housing 207 of the acoustic sensor package 200B. The sampling channel 236 provides a conduit for samples of breathing gases flowing in the lumen 220 to be drawn or siphoned into the sample tube lumen 244, and further transmitted through the gas sample tube 242 to one of the described receiving devices for analysis. In this example configuration the sampling channel 236 intersects the acoustic package lumen 220 distally from the sound-sensitive elements of the acoustic receivers located within the sensor tube 228. The sampling channel opening 237 may be kept small, to minimize the impact on acoustic pulses propagating within the acoustic package lumen 220. In other examples, however, the sampling channel 236 may be positioned proximal the acoustic elements to reduce interference with the acoustic functions caused by the discontinuity in the lumen 220 formed by the sampling channel 236.

In the example depicted, the sampling channel 236 is also angled such that gas traveling through the sampling channel 236 flows partially in the distal-to-proximal direction. Such an angled sampling channel 236 may assist in better capturing gases that are exhaled by the patient.

In examples where the gas sample measurement port 240 is used as part of a capnometry monitoring system, the configuration illustrated in FIG. 2B may be considered a sidestream monitoring configuration. In sidestream capnography, samples of breathing gases are drawn or siphoned from the bulk of breathing gases flowing through the patient breathing circuit and, in this case, through the lumen 220. Sidestream monitoring configurations may greatly reduce the weight and bulk of tubing, and associated dead space, introduced by mainstream monitoring configurations. The sidestream monitoring also provides less interference with the acoustic functions of the example acoustic sensor package 200B.

In other examples, the gas-sampling measurement port 240 may be implemented at other locations along the exterior of the example acoustic sensor package 200B. For instance, the gas sample measurement port 240 may be implemented as part of the design of the nozzle 204. Alternatively, the gas sample measurement port 240 may be located on any side of the example acoustic sensor package 200B, or the sampling channel 236 may be routed through the acoustic package sensor housing 207 in various configurations.

FIG. 2C depicts a cross-sectional view of an example acoustic sensor package 200C, which includes another example configuration of the gas-sampling measurement port 240. Similar to the configuration depicted in FIG. 2B, the gas-sampling tube 242 in FIG. 2C is connected to the gas-sampling measurement port 240. The sampling channel 236 provides a conduit for sampled gas to flow from the lumen 220 to the sampling tube lumen 244. In this example configuration, the gas samples are drawn or siphoned from the bulk of breaking gases through a sampling post or mast 250, which is surrounded by a sampling shroud 252. The position of the sampling mast 250 within the center of the lumen 220 may allow breathing gases travelling at a higher velocity to be drawn or siphoned into the sampling mast 250 and into the sampling channel 236, which may increase gas sampling efficiency. The sampling shroud 252 may reduce turbulent gas flow in the vicinity of the sampling mast 250, which may also improve the quality of breathing gas sampling.

To facilitate the connection between the sampling mast 250 and the sampling channel 236, in some examples, supplemental channel housing 246 may be used within the speaker cavity 224 or in other areas of the example acoustic sensor package 200C that contain voids. The supplemental channel housing 246 may provide additional enclosure material to support or control the flow of sampled breathing gases through sampling channel 236. For instance, to the extent the sampling channel 236 extends through a cavity of the housing 207 (such as the speaker cavity 224) the supplemental channel housing 246 may be additional tubing to define the sampling channel 236.

In some examples, the speaker cavity 224, or other areas of the fitting 202, may not have sufficient volume for accommodating both the elements of the gas sampling feature and the sensors 210, 212, 214, and 216. In such examples, one or more of the sensors 210, 212, 214, or 216 may be positioned in other regions adjacent to the acoustic package lumen 220, such as in the sound sensing section 205. For instance, in the example configuration depicted in FIG. 2C, sensors 210 and 216 are located distally from the sound-sensitive elements located within the sensor tube 228. In this location, the sensors 210 and 216 may have a more significant impact on the acoustic pulses received by the sound-sensitive elements 209. As described above, the sensors 210 and 216 may be kept flush with, or may exhibit minimal protrusion from, the acoustic package lumen wall 221, or the sensors 210 or 216 may be minimally recessed from the acoustic package lumen wall 221. If the presence of the sensors has minimal impact on the received acoustic pulses, the corresponding effects on the transduced signals may be accounted for during processing and/or calibration of the received signals.

The sensors 210 and 216 may be any of a temperature sensor, humidity sensor, pressure sensor, and flow sensor, or may represent other types of sensors provided within the example acoustic sensor package 200C. Sensors 212 and 214 may be any of the remaining sensors described. In further examples, sensors 210 and 216 may be chosen from among the selection of sensors based on size, form factor, or expected impact on the acoustic pulses, among other factors.

FIG. 2D depicts a cross-sectional view of an example acoustic sensor package 200D, which includes an example configuration of a nebulizer injection port 260 (such as nebulizer port 16). One end of a nebulizer injection tube 262 is connected to the nebulizer injection port 260 and the other end of the nebulizer injection tube 262 is connected to a nebulizer (such as nebulizer 12). In examples, the nebulizer may be any of a multitude of medical devices or other apparatus capable of delivering inhalable medicine, therapeutics, anesthesia, or any other type of therapy that may be transmitted through the nebulizer injection tube 262, and ultimately to the patient. The injectant 266 supplied by the nebulizer flows from the nebulizer through the nebulizer injection tube lumen 264 and into an injection channel 268 defined by the housing 207 of the example acoustic sensor package 200D. The injection channel 268 connects to an injection nozzle 270, where the injectant 266 is directed into the lumen 220 and into the breathing gases contained therein. As an example, the injectant 266 may include a pressurized fluid that has been pressurized by a pump of the nebulizer. The pressurized fluid travels through the injection channel 268 and when the pressurized fluid reaches the injection nozzle 270, the pressurized fluid is aerosolized or atomized by the injection nozzle 270 to form the injection spray 272. By positioning the injection nozzle 270 within the example acoustic sensor package 200D rather than at a more proximal position of the breathing circuit, less of the injectant is likely to be lost due to adhesion to the surface of the breathing circuit or via bias flow past a patient wye.

In examples, the injection nozzle 270 may direct the injection spray 272 in a distal direction (toward the patient) at a shallow angle. For instance, the injectant 266 flows through the injection channel 268 at least partially in a proximal-to-distal direction. In some examples, the injection nozzle 270 may be positioned flush with the acoustic package lumen wall 221. In other examples, the injection nozzle 270 may protrude slightly (e.g., less than 2 mm) from the acoustic package lumen wall 221 and into the space of the lumen 220, or the injection nozzle 270 may be recessed from the acoustic package lumen wall 221.

In some examples, the injectant 266 supplied by the nebulizer may be in the form of a vapor, while in other examples the injectant 266 may be in the form of a liquid or aerosol. In examples where the injectant 266 is in gaseous or vaporous form, the example acoustic sensor package 200D may not be configured with an injection nozzle 270, or the injection nozzle may minimally affect the passage of the injectant 266 into the acoustic package lumen 220 (such as by merely changing the direction of the flow of the injectant 266). In examples where the injectant 266 is liquid or aerosol-based, the injection nozzle 270 may be configured to render the liquid or aerosol-based injectant 266 into finer particles for transmission into the acoustic package lumen 220. For example, the injection nozzle 270 may be an atomizer, which converts a liquid form of injectant 266 into a fine spray of liquid mist. In other examples, the injection nozzle 270 may limit the flow of injectant 266 (of any form) into the breathing gases contained within the acoustic package lumen 220.

In examples, the nebulizer injection port 260 may be the same type of tubing connection port as the gas-sampling measurement port 240 (e.g., a Luer connection, any other type of standardized tubing connection, or a custom type of tubing connection). In other examples, the nebulizer injection port 260 may be a different type of tubing connection port than the gas sampling-measurement port 240. In some example configurations, an acoustic sensor package may provide both a nebulizer injection port 260 and gas-sampling measurement port 240, or may provide nether port (such as in example acoustic sensor package 200A). In other configurations, an acoustic sensor package may provide additional sensors or instrumentation (such as temperature, humidity, pressure, flow, or other type of sensor) to monitor the characteristics or properties of injectants 266 or sampled gases passing through the nebulizer injection port 260, the injection channel 268, the gas sample measurement port 240, the sampling channel 236, or other feature of the acoustic sensor package capable of sampling from the breathing gases or transmitting injectant 266 into the breathing gases.

FIGS. 2A, 2B, 2C, and 2D depict a limited number of example acoustic sensor packages 200A, 200B, 200C, and 200D. It should be understood that a wide range of design alternatives exist for implementing the sensor and tube connection port integration described above. For example, the sensor elements are depicted as being located one after another along a proximal-distal axis. In other examples, the sensors may be distributed circumferentially around the lumen 220 or may be arranged in other locations, groupings, or configurations within an acoustic sensor package. In still other examples, the dimensions of the example acoustic sensor packages 200A, 200B, 200C, or 200D may be modified to accommodate different design alternatives for sensor or tube connection port placement.

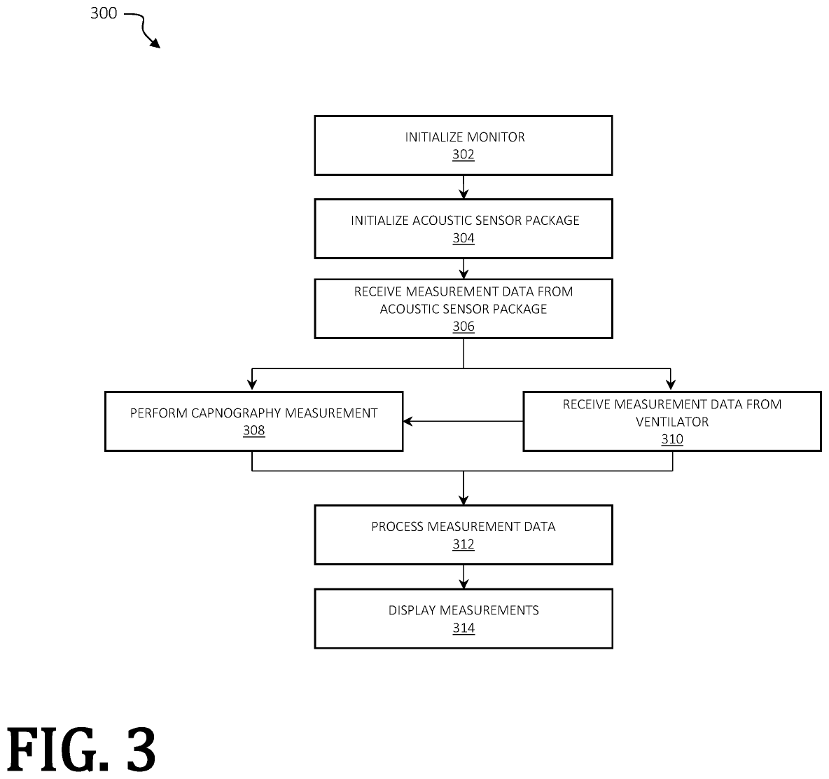

FIG. 3 depicts an example method 300 depicts an example method for capturing measurement data from an acoustic sensor package. At operation 302, the monitor (such as monitor 82) is initialized. The initialization may include powering on the monitor, calibration of instrumentation, circuitry, other electronics, or other electrical or mechanical mechanisms within the monitor. In examples, the initialization may include establishing communication with devices that are communicatively coupled to the monitor. The initialization may include receiving user inputs from a clinician or other user to configure the monitor for operation, or to configure devices connected to the monitor (such as acoustic sensor package 26 or ventilator 22). Such configuration or user input may include setting operating ranges, enabling sensor features or functions, setting circuit operating parameters (such as programmable filtering or amplification), selecting sampling rates, data rates, or setting other selectable operating features provided by the monitor and/or acoustic sensor package. In some examples, the initialization may occur before or after the patient is intubated and a complete breathing circuit is established.

At operation 304, the acoustic sensor package (such as acoustic sensor package 26 or example acoustic sensor packages 200A, 200B, 200B, or 200D) is initialized. The initialization may include calibration of instrumentation, circuitry, other electronics, or other electrical or mechanical mechanisms within the acoustic sensor package. In examples, the initialization may include establishing communication with devices that are communicatively coupled to the acoustic sensor package, such as the monitor (e.g., monitor 82). In examples, the initialization may further include connection of tubing associated with the ETT (such as tracheal tube 30), breathing circuit (such as patient circuit 44), nebulizer (such as nebulizer tube 14), or monitor connection (such as sample tube 94). The initialization of the acoustic sensor package may also include receiving configuration information or other user input from the monitor, where such configuration information or user input may include setting operating ranges, enabling sensor features or functions, setting circuit operating parameters (such as programmable filtering or amplification), selecting sampling rates, data rates, or setting other selectable operating features provided by the acoustic sensor package.

Once the monitor and acoustic sensor package are initialized, at operation 306, the monitor receives measurement data from the acoustic sensor package. As discussed above, the monitor may receive measurement data from the elements of the acoustic sensor package associated with the acoustic generator or acoustic receiver (such as acoustic generator 50 or acoustic receiver 52). This acoustic measurement data may include data associated with airway diameter in the vicinity of the distal tip of the ETT (such as the distal end 62 of tracheal tube 30); displacement of the distal tip of the ETT further into the patient's airway or towards the patient's oral cavity; extubations; size and location of ETT blockages; sound speed of the acoustic pulses between sound-sensitive elements; sound speed of the acoustic pulses between the acoustic generator and one of the sound sensitive elements of an acoustic receiver; or other data associated with characteristics of the patient's airway or ETT. Additionally, the monitor may receive sensor measurement data provided by any sensor present in the acoustic sensor package, such as a temperature, humidity, pressure, flow, or any other type of sensor that may be present (such as sensors 210, 212, 214, and 216).

In some examples, the measurement data may be received in a digital format. For example, the data may be received in a hexadecimal format or other type digital encoding or representation, and the digital data may be grouped into defined data packet structures. In other examples, the measurement data may be received as analog signals. For example, the measurement data may be analog signals received directly from one or more sensing elements or mechanisms, or may be analog signals conditioned by circuitry associated with any of the sensing elements or mechanisms provided within the acoustic sensor package. The measurement data may be received on a continual basis or may be received on an intermittent basis, such as on a periodic basis. In other examples, transmission of measurement data may be controlled by the monitor, where the monitor may indicate when the acoustic sensor package should transmit the measurement data. In such an example, the monitor may request data on a fixed periodic basis, in bursts, asynchronously with other processes, or on-demand. In examples, the transmission of data between the monitor and acoustic sensor package may be configured by the user or may be set by the device manufacturer.

The monitor may also provide a means of performing capnometry analysis. As described, in some examples, the monitor may be coupled to the acoustic sensor package by way of a gas-sampling tube (such as sample tube 94), which provides samples of breathing gases flowing through the sensor acoustic package or other portions of the patient breathing circuit. In examples where the monitor is equipped to perform capnometry measurements (such as by a capnometry analyzer 88) based on gas sampling, the monitor performs such measurements at operation 308. The monitor may use IR-based measurement techniques to determine CO₂ concentration or may use any other established method to perform the measurement on the sampled breathing gases.

Additionally or alternatively, the combination of features provided by the acoustic sensor package and monitor may allow for sound-speed-based capnometry analysis. This type of analysis may be performed by determining the acoustic pulse transit time between two or more acoustic receivers housed within the acoustic sensor package. The presence of CO₂ may affect the speed of travel of the acoustic pulses and these changes may be measurable by the two or more acoustic receivers (e.g., the first sound-sensing element 209A and the second sound-sensing element 209B). In other instances, the pulse transit time between the acoustic generator and an acoustic receiver may be used to determine speed of travel of the acoustic pulses, which may then be used to perform capnometry. The analysis of acoustic pulse transit time may use additional breathing gas measurements or variables, such as temperature, humidity, and/or O₂ concentration. In some examples, the monitor may receive measurement data associated with these breathing gas parameters from the acoustic sensor package, as described for operation 306. In other examples, the monitor may receive measurement data associated with these breathing gas parameters from the ventilator (such as ventilator 22) or from other medical devices or related equipment. At operation 310, the monitor receives measurement data from the ventilator, which may be used in sound-speed-based capnometry. In examples, the monitor may perform capnometry analysis based on breathing gas samples or on acoustic pulse sound speed data. In other examples, the monitor may use a combination of both breathing gas samples and acoustic pulse sound speed data to perform capnometry analysis.

At operation 312, the monitor processes the received measurement data. The measurement data may be in the form of transduced sensor signals, which may or may not have been conditioned by any circuitry associated with the sensor, or the measurement data may be in a digital format. The received measurement data may be processed by any combination of analog or digital circuitry, which may include PCB-mounted, discrete components. Examples of processing performed by this type of circuitry may include filtering, amplifying, level-shifting, analog-to-digital conversion, logical operations (such as by combinational or sequential logic), encoding or decoding, multiplexing, or other types of analog or digital functions.

Additionally or alternatively, the measurement data may be processed by integrated circuits, such as one or more microcontrollers, microprocessors, DSPs, programmable circuits, ASICs, or similar elements. Processing of the received measurement data may include the use of software, firmware, or other executable code. In examples, processing of received measurement data may include application of digital filtering, amplification, frequency analysis, phase analysis, or other types of basic or advanced digital signal processing techniques. In examples, the measurement data processing may include the computation of statistical data measures, such as mean, minimum, maximum, average, standard deviation, or similar metrics, or may include the determination of statistical distributions and associated parameters. In other examples, the measurement data processing may include little or no additional processing of received measurement data by the monitor.

In some examples, measurement data processing may occur on a continual basis, while in other examples measurement data processing may be performed intermittently, as data is received from the acoustic sensor package, ventilator, or other source of measurement data that may be in communication with the monitor. In examples, measurement data processing may include any combination of the above described processing techniques.

At operation 314, the monitor displays any combination of output from the above described measurement data processing. In examples, the display of measured data may include any combination of sensor and acoustic data. The display of measured data may include any of the statistical measures computed by the monitor, including statistical values, statistical distributions, or related data. In examples, the display of measured data may comprise any combination of processed or unprocessed data, in numeric form, graphical form, or both.

The measured data is displayed on a screen, such as an LCD, LED, OLED, or other display technology. In examples, the screen may comprise a touch sensitive display capable of receiving input from the user. In other examples, the display of measured data may also include any of a wide variety of visual indicators such as lamps, bulbs or strip light sources, or other type of discrete visual indicators.

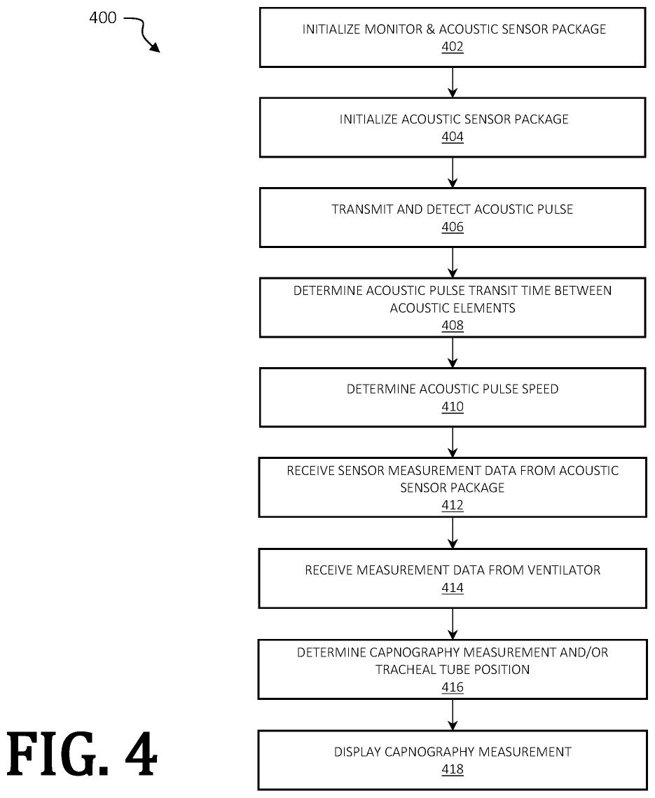

FIG. 4 depicts an example method 400 for performing capnometry using acoustic sound speed data. At operation 402, the monitor (such as monitor 82) is initialized. At operation 404, the acoustic sensor package (such as acoustic sensor package 26 or example acoustic sensor packages 200A, 200B, 200B, or 200D) is initialized. The initialization operations 402, 404 may be substantially the same as the initialization operations 302, 304 discussed above.

At operation 406, an acoustic generator (such as acoustic generator 50 or speaker 208) emits acoustic energy (e.g., an acoustic pulse) into a lumen disposed within the acoustic sensor package for the passage of breathing gases (such as acoustic package lumen 220). The emitted acoustic energy may take the form of an acoustic pulse, sound, or other forms of propagating acoustic energy. The emitted acoustic pulse propagates in a proximal-to-distal direction, past the sound-sensitive element of a first acoustic receiver, then past the sound-sensitive element of a second acoustic receiver. The acoustic pulse continues to propagate distally into the ETT, then into the airway of the patient. The acoustic pulse interacts with the end of the ETT and the patient's anatomy, is reflected back into the ETT, and subsequently travels back into the lumen of the acoustic sensor package. The reflected pulse (now propagating in a distal-to-proximal direction) first passes the sound-sensitive element of the second acoustic receiver, followed by the sound-sensitive element of the first acoustic receiver. The received pulse is transduced by the sound-sensitive elements and the corresponding signals are processed by electronics associated with the sound-sensitive elements (such as those located on PCB 231).

At operation 408, the acoustic pulse transit time between acoustic elements is determined. For instance, a transit time from the speaker to an acoustic receiver may be determined and/or a transit time between two acoustic receivers may be determined. Where the transit time between the speaker and the acoustic receiver is used, the time that the acoustic pulse was emitted by the speaker is known as it was initiated by the device. that emission time and the time the emitted acoustic pulse is detected by the acoustic receiver may then be used to calculated the transit time.

In examples where the transit time is calculated between the two acoustic receivers, either the emitted acoustic pulse, reflected acoustic pulse, or both pulses may be used to determine the pulse transit time. As a propagating acoustic pulse passes the sound-sensitive element of an acoustic receiver, an electric signal is transduced by the acoustic receiver. In examples. the transduced signal may be processed in the analog signal domain (such as by analog circuitry), may be digitized for analysis in the digital domain, or may be processed in any combination of both analog and digital domains. Determination of the pulse transit time may include the use of precision clock sources, counters, or other timing elements or mechanisms. In some examples, the pulse transit time may be determined by signal processing elements housed within the acoustic sensor package, while in other examples, the pulse transit time may be determined by signal processing elements associated with other devices, such as the monitor.

In some examples, determination of pulse transit time may include detecting the time at which the transduced signal at a first acoustic receiver exceeds a pre-determined amplitude threshold, and comparing that time to the same event detected at a second, spatially separated acoustic receiver. In other examples, a variety of signal comparison methods may be performed to determine the timing difference between arrival of an acoustic pulse at one acoustic receiver, relative to the arrival of the same acoustic pulse at a second, spatially separated acoustic receiver.

At operation 410, the acoustic pulse speed between the respective acoustic elements (e.g., the speaker and the acoustic receiver or the first and second acoustic receivers) is determined using the acoustic pulse transit time and known spatial separation of the two acoustic receivers. This calculation may be performed by elements within the acoustic sensor package or within the monitor, by taking the distance between the two acoustic receivers (which is known and fixed by design the acoustic sensor package) and dividing by the pulse transit time determined in operation 408.

At operation 412, the monitor receives measurement data collected by the acoustic sensor package from a plurality of the sensors discussed above, several of which may provide measurement data that may be used in determining CO₂ concentration. For example, both temperature, humidity, and gas flow rate affect the speed of propagating sound waves (such as acoustic pulses), and by extension, affect calculation of CO₂ concentration based on the sound speed. Thus, in examples, data from the acoustic sensor package temperature and humidity sensors may be included in the capnometry analysis, among other sensor data. The sensor measurement data may be received from the acoustic sensor package by the monitor by the means described above, such as by wired electrical connection (e.g., interface cable 96), or by wireless means if such a feature is provided by the monitor and acoustic sensor package.

The propagation of sound waves in a gaseous medium is also affected by the composition of the gas. The major components found in breathing gases are O₂, CO₂, and nitrogen. The concentration of nitrogen in breathing gases may be relatively constant, and may be accounted for without measurement of the nitrogen concentration. The O₂ concentration in the breathing gases, however, may vary and be controlled by the ventilator delivering the breathing gases. Thus, in examples, the O₂ concentration of the breathing may be included in the capnometry analysis. As described, the O₂ concentration is controlled and/or measured by ventilators, and at operation 414, O₂ concentration and/or other measurement data collected by the ventilator may be communicated from the ventilator to the monitor (such as by ventilator cable 98). In some examples, some gas properties, such as pressure, flow, humidity, and/or temperature, may also be received from the ventilator or from sources other than acoustic sensor package (such as sensors on the breathing circuit, in a humidifier, etc.).

Thus, with a substantially constant nitrogen assumption, a measured or known O₂ concentration, temperature, humidity, and/or flow, changes in sound speed can be attributed to changes in CO₂ concentration of the gases flowing through the acoustic sensor package. Accordingly, changes in CO₂ concentration can be calculated. In examples where the acoustic sensor package is sufficiently calibrated, the absolute values of CO₂ concentration may also be determined or estimated.

At operation 416, the measurement data described above may be combined and processed by the monitor (such as by processor 80 or capnometry analyzer 88) to determine CO₂ concentration and/or positioning of the tracheal tube. For example, the same acoustic pulse that is used for determining transit time and speed may also be used for determining the position of the tracheal tube.

In examples, the acoustic planar wave propagation speed; the temperature, humidity, gas flow rate, and O₂ concentration of the breathing gases; and/or other factors, may be used during processing by the monitor. In further examples, the monitor may compute EtCO₂ based on the received measurement data. For instance, based on the pressure and/or flow rates of the gases, the breathing phases may be determined (e.g., the inspiratory phase and the expiratory phase). In such examples, the breath phase data and the determined CO₂ data may be used to determine an EtCO₂ value.

In some examples, the measurement data processing may include the computation of statistical data measures, such as mean, minimum, maximum, average, standard deviation, or similar metrics, or may include the determination of statistical distributions and associated parameters. In further examples, the measurement data processing may include performing a running average of CO₂ concentration over a user-specified or predetermined interval. In some examples, during processing the monitor may determine that the CO₂ concentration is outside a predetermined or user-specified limit, and may indicate an alert or alarm to activated on the monitor or ventilator. The monitor may determine CO₂ concentration on a continual basis, as new measurement data is received, or in other examples, may determine CO₂ concentration on an intermittent basis, wherein the received measurement data is stored (such as in memory 92) and retrieved for analysis on a less-frequent or on-demand basis.

At operation 418, the CO₂ concentration is displayed by the monitor (such as by display 84). The displayed CO₂ concentration may include a numeric representation of CO₂ concentration, graphical representation of CO₂ concentration, or both. In examples where the monitor determines that the CO₂ concentration is outside a predetermined or user-specified limit, the display may indicate a visual or audible alert. In some examples, the display may comprise a screen, such as an LCD, LED, OLED, or other display technology (such as a touch screen display), while in other examples the display may include any type of visual indicator such as lamps, bulbs or strip light sources, or other types of discrete visual indicators. For example, on a more basic type of display, the monitor may illuminate a single LED light source for CO₂ concentration that is in-range and may illuminate a different LED light source for CO₂ concentration out-of-range. Additional data may also be displayed on the screen, such as gas property data and/or tracheal tube position data. In some examples, the combinations of data may be displayed concurrently on the screen.

Those skilled in the art will recognize that the methods and systems of the present disclosure may be implemented in many manners and as such are not to be limited by the foregoing aspects and examples. In other words, functional elements being performed by single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. An acoustic sensor device for connection to a tracheal tube, the acoustic sensor device comprising:
a proximal end connectable to a breathing circuit to receive breathing gases;
a distal end connectable to the tracheal tube;
a housing, between the proximal end and the distal end, defining a lumen through which the breathing gases flow;
an acoustic generator within the housing and positioned to emit acoustic pulses into the lumen;
an acoustic receiver within the housing and positioned distally from the acoustic generator; and
a first gas property sensor within the housing and positioned proximally from the acoustic receiver, the first gas property comprising at least one of a flow sensor, a pressure sensor, a humidity sensor, or a temperature sensor.

2. The acoustic sensor device of claim 1, wherein:
the housing defines a speaker cavity; and
the acoustic generator and the first gas property sensor are disposed within the speaker cavity.

3. The acoustic sensor device of claim 1, further comprising a second gas property sensor positioned distally from the acoustic receiver.

4. The acoustic sensor device of claim 3, wherein the first gas property sensor and the second gas property sensor are recessed into a wall of the lumen.

5. The acoustic sensor device of claim 1, further comprising a sampling channel defined by the housing, the sampling channel including an opening to the lumen.

6. The acoustic sensor device of claim 1, further comprising:
an injection channel defined by the housing, the injection channel; and
an injection nozzle coupled to the injection channel.

7. A method for capturing measurement data from an acoustic sensor device coupled to a tracheal tube, the method comprising:
emitting, from an acoustic generator of the acoustic sensor device, an acoustic pulse into a lumen of the acoustic sensor device;
detecting, by an acoustic receiver of the acoustic sensor device, the acoustic pulse; and
based on the detected acoustic pulse, determining a position of the tracheal tube and a carbon dioxide concentration of breathing gases flowing through the lumen of the acoustic sensor device.

8. The method of claim 7, further comprising:
receiving, from one or more sensors of the acoustic sensor device, temperature and humidity data for the breathing gases; and
wherein determining the carbon dioxide concentration is further based on the temperature and humidity data.

9. The method of claim 7, further comprising:
receiving, from a sensor of the acoustic device or a ventilator, flow rate data for breathing gases flowing through the lumen of the acoustic sensor; and
wherein determining the carbon dioxide concentration is further based on the flow rate data.

10. The method of claim 7, further comprising:
based on data from the acoustic receiver, determining breath phase data; and
based on the breath phase data and the determined carbon dioxide concentration, determining an end-tidal carbon dioxide value.

11. The method of claim 7, further comprising:
determining a speed of the acoustic pulse based on a transit time of the acoustic pulse between the acoustic receiver and another acoustic receiver of the acoustic sensor device; and
wherein the wherein determining the carbon dioxide concentration is further based on the speed of the acoustic pulse.

12. A system for detecting breathing gas properties and tracheal tube positioning, the system comprising:
an acoustic sensor device comprising:
a proximal end connectable to a breathing circuit to receive breathing gases;
a distal end connectable to the tracheal tube;
a housing, between the proximal end and the distal end, defining a lumen through which the breathing gases flow;
an acoustic generator within the housing and positioned to emit acoustic pulses into the lumen;
a first acoustic receiver and a second acoustic receiver within the housing and positioned distally from the acoustic generator;
a first gas property sensor within the housing and positioned proximally from the first acoustic receiver and the second acoustic receiver, the first gas property comprising at least one of a flow sensor, a pressure sensor, a humidity sensor, or a temperature sensor; and
a second gas property sensor within the housing and positioned proximally from the first acoustic receiver and the second acoustic receiver, the second gas property comprising at least one of a flow sensor, a pressure sensor, a humidity sensor, or a temperature sensor; and
a monitor communicatively coupled to the acoustic sensor device, the monitor comprising:
a display;
a processor; and
memory storing instructions that when executed by the processor cause the monitor to perform operations including:
receiving gas property sensor data from the first gas property sensor and the second gas property sensor;
receiving acoustic data captured by the first acoustic receiver and the second acoustic receiver;
displaying, on the display, gas property values based on the received gas property sensor data; and
displaying, on the display, tracheal tube position based on the received acoustic data.

13. The system of claim 12, wherein:
the acoustic sensor device further comprises a sampling channel defined by the housing, the sampling channel including an opening to the lumen; and
the system further comprises a sampling tube pneumatically coupled to the monitor and the sampling channel.

14. The system of claim 13, wherein:
wherein the monitor further comprises a capnography analyzer positioned to receive gas sampled through the sampling tube; and
the operations further comprise:
analyzing the sampled gases to determine a carbon dioxide concentration; and
displaying, on the display, the carbon dioxide concentration.

15. The system of claim 12, wherein:
the acoustic sensor device further comprises an injection channel defined by the housing, the injection channel; and
an injection nozzle coupled to the injection channel.
